(19) United States
(12) Patent Application Publication
(10) Pub. No.: US 2024/0238432 A1
(43) Pub. Date: Jul. 18, 2024

(54) METHOD FOR GLYCOPROTEIN MODIFICATION

(71) Applicant: HoneyBear Biosciences Inc., Taipei City (TW)

(72) Inventors: Shih-Hsien CHUANG, Taipei City (TW); Yu-Wei LAI, Taipei City (TW); Cheng-Chou YU, Taipei City (TW); Shu-Ping YEH, Taipei City (TW); Jin-Yu WANG, Taipei City (TW); Shih-Chong TSAI, Taipei City (TW); Wei-Ting SUN, Taipei City (TW); Chin-Yi Huang, Fremont, CA (US)

(21) Appl. No.: 18/400,973

(22) Filed: Dec. 29, 2023

Related U.S. Application Data
(60) Provisional application No. 63/477,672, filed on Dec. 29, 2022.

(51) Int. Cl. A61K 47/68; A61P 35/00; C12N 9/24; C12P 21/00

(52) U.S. Cl. CPC A61K 47/68031; A61K 47/68037; A61K 47/6889; A61P 35/00; C12N 9/2402; C12P 21/005; C12Y 302/01096

(57) ABSTRACT

A method for modifying glycoproteins is provided. The present disclosure also provides a method for producing glycoprotein-payload conjugates, the conjugates produced thereby, and the use thereof.

This application claims the benefit of priority under 35 USC § 119(e) of U.S. Provisional Patent Application No. 63/477,672 filed on Dec. 29, 2022, the content of which is incorporated by reference as if fully set forth herein in its entirety.

### BACKGROUND

### Technical Field

The present disclosure relates to a method for conjugating payloads of interest to glycoprotein including antibodies and their derivatives to generate glycoprotein-payload conjugates.

### Description of the Related Art

Therapeutic protein drugs have the advantages of high specificity and low toxicity, and have been widely used in clinics since 1990. Among these, over 80% are monoclonal antibodies. While some of the patients are reported with improved clinical outcomes, however, several clinical trials fall short due to unsatisfactory therapeutic efficacy, particularly for cancer treatments.

In an effort to raise the therapeutic efficacy for cancer treatment, scientists focus on modifying the clinically approved monoclonal antibodies by technologies including bispecific antibodies, antibody-drug conjugates (ADC) and CAR-T. Among these, ADC draws more attentions due to higher specificity and efficacy. So far, there are 11 therapeutic antibody-drug conjugates available in the clinics, including Mylotarg, Adcetris, Besponsa, Kadcyla, Polivy, Padcev, Enhertu, Trodelvy, Blenrep, Zynlonta and Aidexi, while many others are under development.

In these early clinical ADCs, most of them have their payloads randomly conjugated to the lysine sites, such as Kadcyla and Mylotarg, or to the cysteine sites such as Adcetris. For example, in Adcetris, an average of 4 (ranging from 2 to 8) monomethyl auristatin E (MMAE) molecules, which are synthetic anti-neoplastic agents, are conjugated randomly to a brentuximab antibody scaffold at the —SH groups of cysteine residues by mild reduction of disulfide bonds. The linker components are a thiol-reactive maleimidocaproyl spacer, the dipeptide valine-citrulline linker, and a PABC spacer (Francisco J A, Cerveny C G, Meyer D L, Mixan B J, Klussman K, Chace D F, Rejniak S X et al. cAC10-vcMMAE. Blood 2003, 4,1458-65). These approaches allow easy conjugation of a payload or linker to an antibody, due to the presence of multiple lysine and cysteine residues in an antibody; however, it is difficult to control the drug-to-antibody ratio (DAR) and thus lack of homogeneity of the ADC products and problems with pharmacokinetics (PK) properties and immunogenicity. These ADCs also face challenges in chemistry manufacturing and controls (CMC).

In recent years, site-specific conjugation technologies are developed to solve these disadvantages of the first-generation ADC including Engineered cysteines, Unnatural amino acids (UAAs) engineering, Enzyme-assisted ligation, (such as SMART-Tag and bacterial transglutaminase), Short peptide tags, and Native cysteine rebridging (such as Thio-Bridge), etc. These technologies are able to generate homogeneous ADC products through engineering specific sites of their parent antibody components. For example, Thio-Bridge technology connects the linker and the payload to the partially reduced disulfide bonds of antibodies. On the other hand, SMART-Tag produces an ADC by mutating an adjacent sequence of antibody to become the substrate sequence for a bacteria oxidase. The resulting product of formaldehyde in an antibody is used as the connecting site of linker and payload. As expected, these second-generation ADC technologies are able to generate homogeneous ADC products with unique DARs. However, due to alterations made to the antibody sequence, unwanted immunogenicity may be resulted. Furthermore, these ADC products are still insufficient for PK requirement.

To address the above issues and to enhance the therapeutic efficacy of ADC, glycoengineering gains attention of developers and provides a new approach to ADC platform development. Antibody drug conjugation by glycoengineering has several advantages, including site specificity, diversity, less side effects and high yield. Patents including U.S. Pat. No. 9,504,758B2, U.S. Pat. No. 9,580,511B2, WO 2015/032899A1, U.S. Ser. No. 11/085,062B2, U.S. Pat. No. 8,716,033B2, U.S. Pat. No. 7,416,858B2, and a review article (Bioconjug Chem.; 2015 Nov. 18; 26(11):2070-5) have disclosed various modified glycan moieties for antibody drug conjugations. However, the drug antibody ratios (DAR) and the payload diversity in the disclosed antibody-drug conjugation remain limited in these publications and thus there is still an unmet need in the art to increase the drug antibody ratio and payload diversity of an ADC.

### SUMMARY

The present disclosure relates to a method for conjugating payloads of interest to glycoprotein including antibodies and their derivatives to generate glycoprotein-payload conjugates.

The nature glycan form of monoclonal antibodies comprises the structure below, including GlcNAc¹ bonded to Asn297 of the antibody; GlcNAc² bonded to a first mannose (Man¹); a second and a third mannose (Man² and Man³) respectively bonded to the α-1,3 and α-1,6 positions of Man¹; and two further GlcNAc sugars (GlcNAc³ and GlcNAc⁴) respectively bonded to the β-1,2 positions of Man² and Man³. An antibody having such glycan moieties is represented as GOF, however, when the fucose moiety is absent, the antibody is G0. (T. Shantha Raju MAbs. 2012 May 1; 4(3): 385-391). When one of the GlcNAc³ and GlcNAc⁴ bonds to an additional galactose sugar (Gal), the antibody is represented as G1F/G1 antibody. When both the GlcNAc sugars in the glycan moiety of an antibody respectively bond to two additional galactose sugars (Gal), the antibody is represented as G2F or G2 antibody. Antibodies produced by mammalian cells generally may include G0F (more than 40%), G1F (about 30%-40%) and G2F (less than 1%), and a very small amount of G1F and G2F linking to sialic acid (SA):

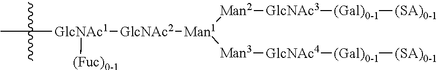

The present disclosure relates to a method for the preparation of a glycoprotein, comprising a core internal mannose (Man) substituent with a compound of the formula GlcNAc-(Q)₀₋₈-C′ in the presence of a suitable catalyst, wherein said catalyst comprises from an acetylglucosaminyltransferase such as beta-1,4-mannosyl-glycoprotein 4-beta-N-acetylglucosaminyltransferase, mannosyl (α-1,3-)-glycoprotein β-1,2-N-acetylglucosaminyltransferase and mannosyl (α-1,6-)-glycoprotein β-1,2-N-acetylglucosaminyltransferase, wherein Q is selected from alkylene, alkenylene or polyehylene glycol and C′ is or comprises a reactive group selected from an azido group, a keto group, an alkynyl group, a thiol group, a halogen, a sulfonyloxy group, a halogenated acetamido group, a mercaptoacetamido group, a sulfonylated hydroxyacetamido group, a cyclopropenyl group, a trans cyclooctene group, a cycloalkyne group, a tetrazyinyl group, a maleimide group, a cyclononyne moiety and a cyclooctyne moiety.

The present disclosure also relates to a method for the preparation of an antibody-conjugate, comprising reacting the modified antibody according to the invention with a linker-payload conjugate, wherein said linker-payload conjugate comprises a reactive group G′ and one or more payloads of interest, wherein said G′ is or comprises a reactive group capable of undergoing a reaction with C′ of a GlcNAc-(Q)₀₋₈-C′.

One aspect of the present disclosure provides method for preparing a glycoprotein-conjugate comprising a structure of formula (1)

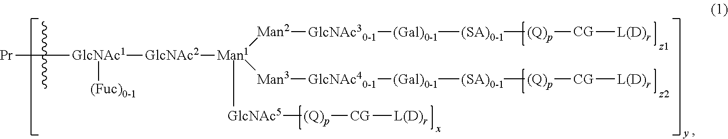

wherein Pr is a glycoprotein; L is a linker; D is a payload of interest; x is 1, 2, 3, or 4; y is 1 or 2; z₁ is 0 or 1; z₂ is 0 or 1; r is a positive integer from 1-20; p is an integer from 0-8; Q is alkylene, alkenylene or polyehylene glycol; CG is a connecting group obtainable by a reaction of C′ and G′; provided that when z₁ is 1, GlcNAc³ is present, and galactose (Gal) and sialic acid (SA) connected thereon do not present; and provided that when z₂ is 1, GlcNAc⁴ is present, and galactose (Gal) and sialic acid (SA) connected thereon do not present.

The nature glycan form of monoclonal antibodies having the following structure of formula (1-1)

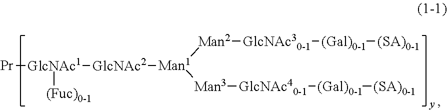

can convert to glycoprotein-conjugate comprising a structure of formula (1) through glycan engineering process with a molecule of UDP-GlcNAc-(Q)₀₋₈-C′ in the presence of beta-1,4-mannosyl-glycoprotein 4-beta-N-acetylglucosaminyltransferase (MGAT3; GnT-3; EC 2.4.1.144) and optionally mannosyl (α-1,3-)-glycoprotein β-1,2-N-acetylglucosaminyltransferase (MGAT1; GnT-1; EC:2.4.1.101) and optionally mannosyl (α-1,6-)-glycoprotein β-1,2-N-acetylglucosaminyltransferase (MGAT2; GnT-2; EC 2.4.1.143) to bond a molecule of GlcNAc-(Q)₀₋₈-C′ to Man′ and optionally Man² and Man³, wherein C′ is or comprises a reative group selected from an azido group, a keto group, an alkynyl group, a thiol group, a halogen, a sulfonyloxy group, a halogenated acetamido group, a mercaptoacetamido group, a sulfonylated hydroxyacetamido group, a cyclopropenyl group, a trans cyclooctene group, a cycloalkyne group, a tetrazyinyl group, a maleimide group, a cyclononyne moiety and a cyclooctyne moiety; and thereby forming a glycoprotein comprising a glycan of formula (1-2):

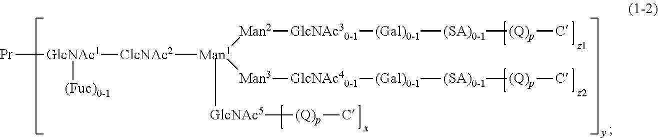

then, reacting the glycoprotein comprising the glycan of formula (1-2) with one or more linker-payload conjugates comprising a structure of G′-L(D)ᵣ; thereby forming the glycoprotein-payload conjugate comprising the structure of formula (1).

In some preferred embodiments, the linker comprises a structure of L₁-L₂-L₃, in which L₁ is present or absent, and when present it is a spacer that may be a linear or branched PEG chain having 1 to 10 PEG units, alkylene, cycloalkylene, alkenylene, cycloalkenylene, alkynylene, arylene, heteroarylene, alkeneoxy, acyl, alkylamines, or arylamine group having 2 to 20 carbon atoms; L₂ is a cleavable linker or a non-cleavable linker that may be a thioether linker, a maleimido caproyl linker, a disulfide containing linker, an acid labile linker, a photolabile linker, a peptidase labile linker, an esterase labile linker, a phosphatase labile linker, a beta-glucuronide linker, a beta-glucuronidase labile linker, a beta-galactosidase labile linker or a sulfatase labile linker; L₃ is present or absent, and when present it is PAB.

In some preferred embodiments, the payload is a therapeutic agent selected from antimetabolites, alkylating agents, alkylating-like agents, DNA minor groove alkylating agents, anthracyclines, antibiotics, calicheamicins, antimitotic agents, topoisomerase inhibitors, proteasome inhibitors, and radioisotopes, for example, the therapeutic agent is selected from exatecan and MMAE; alternatively, the payload is a label selected from a fluorescent label, a chromophoric label, an electron-dense label, a chemiluminescent label, a radioactive label, an enzymatic label, or a positron emitter. In some preferred embodiments, before conducting the step (i) above, a β-N-acetylglucosaminidase may be added to remove GlcNAc on the glycoprotein of formula (1-1).

The invention also relates to a glycoprotein-payload conjugate comprising the structure of formula (1) as defined above. In some preferred embodiments, the glycoprotein-payload conjugate comprises the following structure of formula (2):

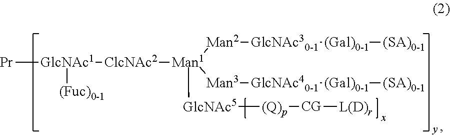

In some preferred embodiments, the glycoprotein-payload conjugate comprises the following structure of formula (3):

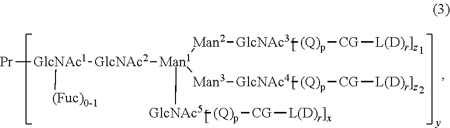

wherein, z₁ and z₂ are both 1, or z₁ is 1 and z₂ is 0.

In some preferred embodiments, the glycoprotein-payload conjugate comprises the following structure of formula (3-1):

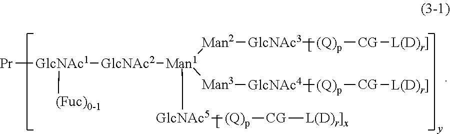

In some preferred embodiments, the glycoprotein-payload conjugate comprises the following structure of formula (3-2):

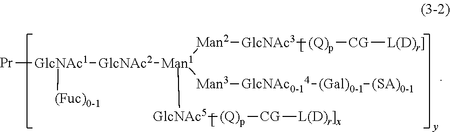

The invention also relates to a method of treating cancer, comprising administrating an effective amount of the glycoprotein-payload conjugate comprising the structure of formula (1), formula (2), or formula (3) as defined above to a subject in need thereof.

The invention also relates to a use of the glycoprotein-payload conjugate comprising the structure of formula (1), formula (2), or formula (3) as defined above in the manufacture of a medicament for treating cancer.

The invention also relates a glycoprotein-payload conjugate comprising the structure of formula (1), formula (2), or formula (3) as defined above for use in treating cancer in a subject in need thereof.

In some preferred embodiments, the cancer is selected from the group consisting of bladder cancer, bone cancer, brain tumor, breast cancer, colorectal cancer, eye melanoma, gastric carcinoma, head and neck cancer, kidney cancer, leukemia, lung cancer, lymphoma, melanoma, oral and oropharyngeal cancer, pancreatic cancer, prostate cancer, skin cancer, stomach cancer, thyroid cancer and uterine cancer, for example, the cancer can be breast cancer and/or gastric carcinoma.

### BRIEF DESCRIPTION OF THE DRAWINGS

The present disclosure will become more readily appreciated by reference to the following descriptions in conjunction with the accompanying drawings.

FIG. 1 shows the reduced mass chromatography of the Herceptin-2Az, which was produced by transferring GlcNAz to Herceptin antibody by GnT-3.

FIG. 2 shows the LC-MS spectrum of Herceptin-2exatecan of the present disclosure, which was produced by conjugating 2 exatecan to the Herceptin-2Az.

FIG. 3 shows the LC-MS spectrum of Herceptin-2MMAE of the present disclosure, which was produced by conjugating 2 MMAE to the Herceptin-2Az.

FIG. 4 shows the LC-MS spectrum of Herceptin-6exatecan of the present disclosure, which was produced by conjugating 6 exatecan to the Herceptin-6Az.

FIG. 5 shows the LC-MS spectrum of Herceptin-6 MMAE of the present disclosure, which was produced by conjugating 6 MMAE to the Herceptin-6Az.

FIG. 6 shows the inhibition effects of Herceptin-2MMAE and Herceptin-6MMAE on the cell viability of breast cancer cell line BT-474.

FIG. 7 shows the inhibition effects of Herceptin-2exatecan and Herceptin-6exatecan on the cell viability of breast cancer cell line BT-474.

FIG. 8 shows the inhibition effects of Herceptin-2MMAE and Herceptin-6MMAE on the cell viability of gastric carcinoma cell line NCI-N87.

FIG. 9 shows the inhibition effects of Herceptin-2exatecan and Herceptin-6exatecan on the cell viability of gastric carcinoma cell line NCI-N87.

### DETAILED DESCRIPTIONS OF THE EMBODIMENTS

Unless defined otherwise, all technical and scientific terms used herein have the same meanings as commonly understood by one of ordinary skill in the art to which this invention belongs. Although any methods and materials similar or equivalent to those described herein can be used in the practice or testing of the present disclosure, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art.

Abbreviations as used in the present disclosure are listed below for reference:

| | |
|---|---|
| ADC | antibody-drug conjugate |
| DAR | drug-to-antibody ratio |
| Asn | asparagine |
| GlcNAc | N-acetylglucosamine |
| GlcNAz | N-azidoacetylglucosamine |
| Gal | galactose |
| Fuc | fucose |
| SA | sialic acid |
| Man | mannose |
| MGAT1; | mannosyl (α-1,3-)-glycoprotein β-1,2-N- |
| GnT-1 | acetylglucosaminyltransferase |
| MGAT2; | mannosyl (α-1,6-)-glycoprotein β-1,2-N- |
| GnT-2 | acetylglucosaminyltransferase |
| MGAT3; | beta-1,4-mannosyl-glycoprotein 4-beta-N- |
| GnT-3 | acetylglucosaminyltransferase |
| UDP | uridine diphosphate |
| DBCO | dibenzocyclooctyne group |
| DM1 | mertansine |
| PEG | polyethylene glyco |
| MMAE | monomethyl auristatin E |

It should be noted that as used herein and in the appended claims, the singular forms “a,” “an,” and “the” include plural reference unless the context clearly dictates otherwise. As well, the terms “a” (or “an”), “one or more” and “at least one” can be used interchangeably herein. It is also to be noted that the terms “comprising,” “including,” and “having” can be used interchangeably.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein the term “about” refers to ±20%, ±15%, ±10%, ±9%, ±8%, ±7%, ±6%, ±5%, ±4%, ±3%, ±2%, ±1%, ±0.5%, or ±0.25%.

When referring to a component of a therapeutic formulation, it is intended that the term used, e.g., “agent,” encompass not only the specified molecular entity but also its pharmaceutically acceptable analogs, including, but not limited to, salts, esters, amides, prodrugs, conjugates, active metabolites, and other such derivatives, analogs, and related compounds.

The general term “sugar” used herein indicates a monosaccharide, for example glucose (Glc), galactose (Gal), mannose (Man) and fucose (Fuc), as well as derivatives of a monosaccharide, such as an amino sugar and a sugar acid, e.g., glucosamine (GlcN), galactosamine (GalN), N-acetylglucosamine (GlcNAc), N-azidoacetylglucosamine (GlcNAz), N-acetylgalactosamine (GlaNAc), N-acetylneuraminic acid (NeuNAc), N-acetlymuramic acid (MurNAc), glucuronic acid (GlcA), and iduronic acid (IdoA).

As used herein, the term “protein” can include a polypeptide having a native amino acid sequence, as well as variants and modified forms regardless of their origin or mode of preparation. A protein which has a native amino acid sequence is a protein having the same amino acid sequence as obtained from nature. Such native sequence proteins can be isolated from nature or can be prepared using standard recombinant and/or synthetic methods. Native sequence proteins specifically encompass naturally occurring truncated or soluble forms, naturally occurring variant forms (e.g., alternatively spliced forms), naturally occurring allelic variants and forms including post-translational modifications. A native sequence protein includes proteins following post-translational modifications such as glycosylation, or phosphorylation, or other modifications of some amino acid residues.

As used herein, the term “glycoprotein” refers to a protein comprising one or more monosaccharide or oligosaccharide chains covalently bonded to the protein. A glycan may be attached to a hydroxyl group of the protein (O-linked-glycosyl), e.g., to the hydroxy goup of serine, threonine, tyrosine, hydroxylysine or hydroxyproline, or to an amide on the protein (N-glycoprotein), e.g., asparagine or arginine, or to a carbon on the protein (C-glycoprotein), e.g., tryptophan. A glycoprotein may comprise more than one glycan, may comprise a combination of one or more monosaccharide and one or more oligosaccharide glycans, and may comprise a combination of N-linked, O-linked and C-linked glycans. Examples of glycoproteins include ligands specific to surface antigens of cells, prostate-specific membrane antigen, Candida antarctica lipase, gp41, gp120, erythropoietin (EPO), antifreeze protein and antibodies.

An antibody is a protein generated by the immune system that is capable of recognizing and binding to a specific antigen. The term antibody herein is used in its broadest sense and specifically includes monoclonal antibodies, polyclonal antibodies, dimers, multimers, multispecific antibodies (e.g. bispecific antibodies), antibody fragments, and double and single chain antibodies. The term “antibody” herein is also meant to include human antibodies, humanized antibodies, chimeric antibodies and antibodies specifically binding to cancer antigen. The term “antibody” is meant to include whole antibodies, but also fragments of an antibody, for example an antibody Fab fragment, F(ab′)2, Fv fragment or Fc fragment from a cleaved antibody, a scFv-Fc fragment, a minibody, a diabody or a scFv. Furthermore, the term includes genetically engineered derivatives of an antibody. Antibodies, fragments of antibodies and genetically engineered antibodies may be obtained by methods that are known in the art. Examples of antibodies in market include, but are not limited to, abciximab, rituximab, basiliximab, palivizumab, infliximab, trastuzumab, alemtuzumab, adalimumab, tositumomab-1131, cetuximab, ibrituximab tiuxetan, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab and brentuximab.

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both variable domains of heavy chain (VH) and light chain (VL), the VH and VL regions may be expressed using a single vector, e.g., in a di-cistronic expression unit, or under the control of different promoters. In other embodiments, the VH and VL region may be expressed using separate vectors. A VH or VL region as described herein may optionally comprise a methionine at the N-terminus.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity.

Techniques for the production of single chain antibodies or recombinant antibodies (U.S. Pat. Nos. 4,946,778, 4,816,567) can be adapted to produce antibodies to polypeptides of this disclosure. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)).

Naturally occurring IgGs and recombinant antibodies have a N-glycosylation site at amino acid asparagine 297 (Asn297) in each CH2 constant region of the IgG1 heavy chain. Through glycosylation and post translational modification in mammalian cells, two di-antenna-shaped glycan moieties can be formed through the N-glycosylation on an IgG, and each of the glycan moieties is basically constructed by at least 7 sugar moieties of GlcNAc and Man having a structure shown in formula (4):

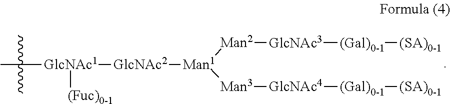

The glycan moiety shown in formula (4) comprise of sugar moieties in a sequence of GlcNAc¹ bonding to Asn297 of an antibody; followed by GlcNAc² which in turn bonds to a first mannose (Man¹); a second and a third mannose (Man² and Man³) bond to the α-1,3 and α-1,6 positions of Man¹ respectively; and two further GlcNAc sugars (GlcNAc³ and GlcNAc⁴) bond to the 3-1,2 positions of Man² and Man³, respectively. An antibody having such glycan moieties is represented as G0, however, when a fucose moiety bond to GlcNAc¹ is present, the antibody is G0F. (T. Shantha Raju MAbs. 2012 May 1; 4(3): 385-391). When one of the GlcNAc³ and GlcNAc⁴ bonds to an additional galactose sugar, the antibody is represented as G1/G1F antibody. When both GlcNAc sugars in the glycan moiety of an antibody bond to two additional galactose sugars respectively, the antibody is represented as G2 or G2F antibody. Antibodies produced by mammalian cells generally include G0F (more than about 40%), G1F (about 30%-40%) and G2F (less than 1%), and a very small amount of G1F and G2F linking to sialic acid.

Glycoengineering of an antibody involving modifications at branching sites of the N297 glycans maintains intact structure and creates functional diversity, while enhances its therapeutic properties such as antibody dependent cell-mediated cytotoxicity (ADCC) and half-life, as well as chemistry manufacturing and controls (CMC) of antibodies.

One aspect of the invention provides a method for producing a glycoprotein conjugate comprising a structure of formula (1):

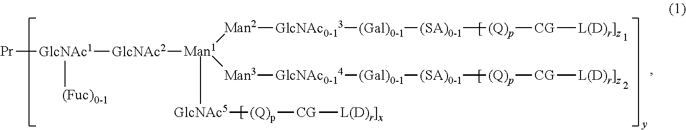

comprising the steps of:
   (i) reacting a glycoprotein comprising a structure of formula (1-1):

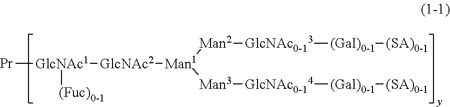

with a molecule of UDP-GlcNAc-(Q)₀₋₈-C′ in the presence of beta-1,4-mannosyl-glycoprotein 4-beta-N-acetylglucosaminyltransferase (MGAT3; GnT-3; EC 2.4.1.144) and optionally mannosyl (α-1,3-)-glycoprotein β-1,2-N-acetylglucosaminyltransferase and optionally mannosyl (α-1,6-)-glycoprotein β-1,2-N-acetylglucosaminyltransferase to bond a molecule of GlcNAc-(Q)₀₋₈-C′ to Man¹ optionally Man² and Man³; and thereby forming a glycoprotein comprising a glycan of formula (1-2)

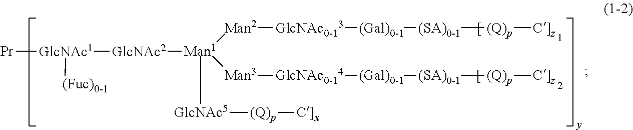

(ii) reacting the modified glycoprotein (1-2) with one or more linker-payload conjugates comprising a structure of G′-L(D)ᵣ; thereby forming the glycoprotein-payload conjugate comprising the structure of formula (1).

In the formula (1), CG is a connecting group obtainable by a reaction of C′ and G′. The term “connecting group” refers to a structural element connecting one part of a compound and another part of the same compound. In the formula (1), CG connects the antibody and payloads, possibly via Q and linker L, if present.

C′ is or comprises a reative group selected from an azido group, a keto group, an alkynyl group, a thiol group, a halogen, a sulfonyloxy group, a halogenated acetamido group, a mercaptoacetamido group, a sulfonylatedhydroxyacetamido group, a cyclopropenyl group, a trans cyclooctene group, a cycloalkyne group, a tetrazyinyl group, a cyclononyne moiety and a cyclooctyne moiety.

Furthermore, said G′ is or comprises a reactive group capable of undergoing a reaction with C′ of a GlcNAc-(Q)₀₋₈-C′. In a preffered embodiment, the G′ comprises a terminal azido, alkyne, cyclononyne moiety or a cyclooctyne moiety, and wherein G′ is different from C′.

For example, the cyclononyne moiety may be bicyclononyne (BCN), and the cyclooctyne moiety may be selected from the group consisting of azadibenzocyclooctyne (DIBAC/DBCO), dibenzocyclooctyne (DIBO) and sulfonylated dibenzocyclooctyne (s-DIBO).

As will be understood by the person skilled in the art, the nature of a connecting group depends on the type of reaction between C′ and G′. For example, when C′ is azido, and the conjugator G′ is alkynyl, the linker-payload reacts with a molecule having a -GlcNAc-(Q)₀₋₈-C′ group to form a -GlcNAc-(Q)₀₋₈-CG-linker-payload through click reaction (Angewandte Chemie International Edition. 40 (11): 2004-2021; and Australian Journal of Chemistry. 60 (6): 384-395). In another embodiment, when C′ is an a ketone group or an aldehyde group, and the G′ is amino, the linker-payload reacts with a molecule having a -GlcNAc-(Q)₀₋₈-C′ group to form a -GlcNAc-(Q)₀₋₈-CG-linker-payload through reductive amination (J. Org. Chem., 2010, 75, 5470-5477; and Synthesis, 2011, 490-496). In a further embodiment, when C′ is an a ketone group or an aldehyde group, and the G′ is β-arylethylamino, the linker-payload reacts with a molecule having a -GlcNAc-(Q)₀₋₈-C′ group to form a -GlcNAc-(Q)₀₋₈-CG-linker-payload through Pictet-Spengler reaction (Bioconjugate Chem., 2013, 24 (6), pp 846-851). Additional suitable combinations of C′ and G′, and the nature of resulting connecting group CG are known to a person skilled in the art.

In the formula (1), Q is alkylene, alkenylene or polyehylene glycol, and Q may or may not exist. In addition, when Q presents, p may independently be 1, 2, 3, 4, 5, 6,7, or 8.

In the formula (1), “-(Fuc)₀₋₁” represents a fucose sugar that is optionally existing, and when it presents, there is only one fucose sugar moiety. Similarly, “-(Gal)₀₋₁” represents a galactose sugar that is optionally existing, and when it presents, there is only one galactose sugar moiety; -(SA)₀₋₁” represents a sialic acid that is optionally existing, and when it presents, there is only one sialic acid moiety; and “GlcNAc₀₋₁” represents a GlcNAc that is optionally existing, and when it presents, there is only one GlcNAc moiety.

In the formula (1), “-[(Q)ₚ-CG-L(D)ᵣ]z₁” represents that a linker-payload, is optionally existing, conjugated on GlcNAc³, and when “-[(Q)ₚ-CG-L(D)ᵣ]z₁” presents, there is only one “-[(Q)ₚ-CG-L(D)ᵣ]” and the galactose sugar and sialic acid on GlcNAc³ do not exist. Similarly, “-[(Q)ₚ-CG-L(D)ᵣ]z₂” represents that a linker-payload, is optionally existing, conjugated on GlcNAc⁴, and when “-[(Q)ₚ-CG-L(D)ᵣ]z₂” presents, there is only one “-[(Q)ₚ-CG-L(D)ᵣ]” and the galactose sugar and sialic acid on GlcNAc⁴ do not exist. “-[(Q)ₚ-CG-L(D)ᵣ]z₁” and “-[(Q)ₚ-CG-L(D)ᵣ]_{z2}” can be both present, both absent, or only “-[(Q)ₚ-CG-L(D)ᵣ]z₁” is present, as shown by the following formula (2), (3-1) and (3-2):

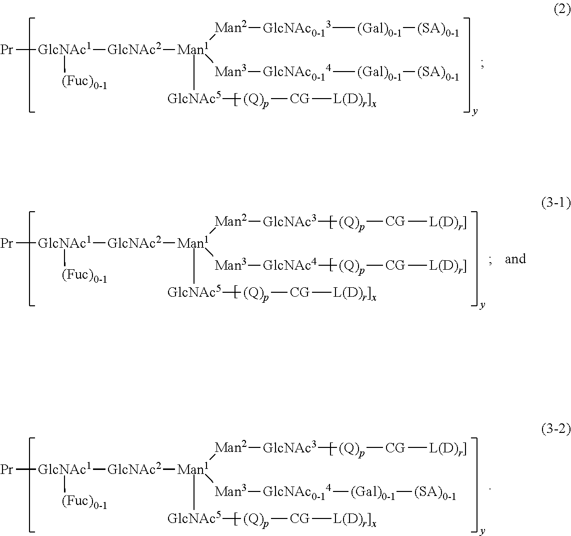

In the formula (1), r is a positive integer within the rage of 1-20, for example, r can be 1, 2, 3, 4, 5, 6, 7, 8, 9, 10, 11, 12, 13, 14, 15, 16, 17, 18, 19 or 20, respectively.

In the formula (1), x is a positive integer within the rage of 1-4, for example, x can be 1, 2, 3 or 4.

In the formula (1), y is 1 or 2.

In some embodiments, the glycoprotein-payload conjugate comprising a structure of formula (1) has the following structure of formula (2):

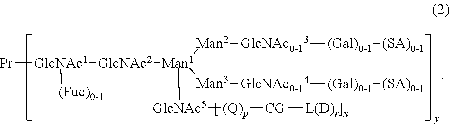

In other embodiments, the glycoprotein-payload conjugate comprising a structure of formula (1) has the following structure of formula (3):

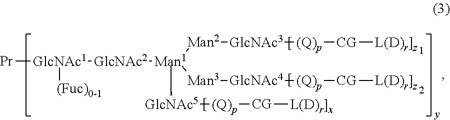

wherein, z₁ and z₂ are both 1, or z₁ is 1 and z₂ is 0.

Glycoproteins as used in the invention may be obtained, for example, by solid-state peptide synthesis (e.g. Merrifield solid phase synthesis) or recombinant production. For recombinant production, one or more polynucleotide encoding the glycoprotein is isolated and inserted into a vector for further cloning and/or expression in a host cell. Such polynucleotide may be readily isolated and sequenced using conventional procedures. Methods which are well known to those skilled in the art can be used to construct expression vectors containing the coding sequence of the glycoprotein. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989).

As used herein, β-N-acetylglucosaminidase belongs to a glycosidase family that catalyzes the hydrolysis of β-N-acetylglucosamine residues from oligosaccharides. Many β-N-acetylglucosaminidases are known to have broard hydrolysis ability in catalyzing multiple types of β-glycosidic linkage. In the present disclosure, the β-N-acetylglucosaminidase is defined as any exoglycosidase that is able (but not limited) to hydrolysis a R 1-2 linkage between terminal acetylglucosamine residues and N-glycan of a glycoprotein. Exo-o-N-acetylglucosaminidase are widely expressed in multiple species, such as Streptococcus spp and Canavalia ensiformis, and are routinely used in identifying terminal GlcNAc from N-glycoprotein.

According to the invention, mannosyl (α-1,3-)-glycoprotein β-1,2-N-acetylglucosaminyltransferase (MGAT1; GnT-1; EC:2.4.1.101) transfers N-acetyl-D-glucosamine from UDP-GlcNAc to a terminal mannose which is linked to another sugar moiety or glycan through alpha 1-3 glycosidic linkage. The linkage of GlcNAc and alpha 3 mannose transferred by MGAT1 is a β1-2 glycosidic bond. It has been found that MGAT1 is universally expressed in eukaryote because it is an essential enzyme to hybrid and complex N-glycan biosynthesis in Golgi.

According to the invention, mannosyl (α-1,6-)-glycoprotein β-1,2-N-acetylglucosaminyltransferase (MGAT2; GnT-2; EC 2.4.1.143) transfers N-acetyl-D-glucosamine from UDP-GlcNAc to a terminal mannose which is linked to another sugar moiety or glycan through alpha 1-6 glycosidic linkage. The linkage of GlcNAc and alpha 6 mannose transferred by MGAT2 is beta 1-2 glycosidic bond. It has been found that MGAT2 is universally expressed in eukaryote because it is an essential enzyme for complex N-glycans biosynthesis in Golgi.

As used herein, beta-1,4-mannosyl-glycoprotein 4-beta-N-acetylglucosaminyltransferase (MGAT3; GnT-3; EC 2.4.1.144) transfers GlcNAc from UDP-GlcNAc and bond to a mannose, where the mannose is bond to another sugar moiety or glycan through a glycosidic linkage. MGAT3 is universally expressed in eukaryote because it is an essential enzyme for hybrid and complex N-glycan biosynthesis in Golgi.

In some embodiments, the reaction between the glycoprotein comprising the glycan having formula (4) and β-N-acetylglucosaminidase is performed in a mammalian cell culture. In the mammalian cell culture, a mammalian cell line, which comprises a first polynucleotide encoding the glycoprotein comprising the glycan having formula (4), and a second polynucleotide encoding the β-N-acetylglucosaminidase, is incubated in a medium at a condition suitable for expression of the glycoprotein and the β-N-acetylglucosaminidase. Examples of mammalian host cell lines include monkey kidney CV1 line transformed by SV40 (COS-7); human embryonic kidney line (293 or 293T cells), baby hamster kidney cells (BHK), mouse sertoli cells (TM4 cells), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells, MRC 5 cells, FS4 cells, Chinese hamster ovary (CHO) cells, and myeloma cell lines such as YO, NSO, P3X63 and Sp2/0.

In some embodiments, when the glycoprotein-payload conjugate is used for treatment of a disease in a subject, the payload may be a therapeutic agent. The therapeutic agent can be a cytostatic or cytotoxic agent or an isotope-chelating agent with corresponding radioisotopes. Examples of the cytostatic or cytotoxic agent include, without limitation, antimetabolites (e.g., fluorouracil (5-FU), floxuridine (5-FudR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, 25 riethylenet, azathioprine, cytosine methotrexate, trimethoprim, pyrimethamine, or pemetrexed); alkylating agents (e.g., cmelphalan, chlorambucil, busulfan, thiotepa, ifosfamide, carmustine, lomustine, semustine, streptozocin, dacarbazine, mitomycin C, cyclophosphamide, mechlorethamine, uramustine, dibromomannitol, tetranitrate, procarbazine, altretamine, mitozolomide, or temozolomide); alkylating-like agents (e.g., cisplatin, carboplatin, nedaplatin, oxaliplatin, satraplatin, or triplatin); DNA minor groove alkylating agents (e.g., duocarmycins such as CC-1065, and any analogs or derivatives thereof; pyrrolobenzodiazapenes, or any analogs or derivatives thereof); anthracyclines (e.g., daunorubicin, doxorubicin, epirubicin, idarubicin, or valrubicin); antibiotics (e.g., dactinomycin, bleomycin, mithramycin, anthramycin, streptozotocin, gramicidin D, mitomycins (e.g., mitomycin C); calicheamicins; antimitotic agents (including, e.g., maytansinoids (such as DM1, DM3, and DM4), auristatins (including, e.g., monomethyl auristatin E (MMAE) and monomethyl auristatin F (MMAF)), dolastatins, cryptophycins, vinca alkaloids (e.g., vincristine, vinblastine, vindesine, vinorelbine), taxanes (e.g., paclitaxel, docetaxel, or a novel taxane), tubulysins, and colchicines); topoisomerase inhibitors (e.g., exatecan, irinotecan, topotecan, camptothecin, etoposide, teniposide, amsacrine, or mitoxantrone); HDAC inhibitor (e.g., vorinostat, romidepsin, chidamide, 25riethylenet, or belinostat); proteasome inhibitors (e.g., peptidyl boronic acids); as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lul⁷⁷. Examples of the isotope-chelating agents include, without limitation, ethylenediaminetetraacetic acid (EDTA), diethylenetriamine-N,N,N′,N″,N″-pentaacetate (DTPA), 1,4,7,10-tetraazacyclododecane-N,N′,N″,N′″-tetraacetate (DOTA), 1,4,7,10-tetrakis(2-hydroxypropyl)-1,4,7,10-tetraazacyclododecane (THP), 26 riethylenetetramine-N,N,N′,N″,N′″,N′″-hexaacetate (TTHA), 1,4,7,10-tetraazacyclododecane-N,N′,N″,N′″-tetrakis(methylenephosphonate) (DOTP), and mercaptoacetyltriglycine (MAG3).

In some embodiments, when the glycoprotein-payload conjugate is used for detection, the payload may be a label. The labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes P³², C¹⁴, I¹²⁵, H³, and I¹³¹, fluorophores such as rare earth chelates or fluorescein and its derivatives, rhodamine and its derivatives, dansyl, umbelliferone, luceriferases, e.g., firefly luciferase and bacterial luciferase, luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. In another embodiment, a label is a positron emitter. Positron emitters include but are not limited to Ga⁶⁸, F¹⁸, Cu⁶⁴, Y⁸⁶, Br⁷⁶, Zr⁸⁹, and I¹²⁴.

As used herein, L is a linker that is capable of being used to couple the connecting group CG to a therapeutic agent or label. In some embodiments, L may contain one or more of L₁, L₂ and L₃. Each of L₁, L₂ and L₃ may be absent or present. In some embodiments, all three linking units are present. In some embodiments, L comprises a structure of L₁-L₂-L₃.

Linker L₁ is a spacer connecting the reactive moieties G′ or connecting group CG with L₂ (when present) or the payload. A “spacer” or spacer-moiety is herein defined as a moiety that spaces (i.e. provides distance between) and covalently links together two (or more) different parts. Spacers that can be used in the linker are know in the art, for example, the spacer used herein can be selected from the group consisting of a linear or branched PEG chain having 1 to 10 PEG units, alkylene, cycloalkylene, alkenylene, cycloalkenylene, alkynylene, arylene, heteroarylene, alkeneoxy, acyl, alkylamines, and arylamine group having 2 to 20 carbon atoms.

In an embodiment, the term “PEG chain” means a polymer comprising repeating “PEG” units of the formula [CH₂CH₂O]ₙ, wherein n is 1 to 10. For example, n may be 1, 2, 3, 4, 5, 6, 7, 8, 9 or 10. In an embodiment, the term “PEG1-10” means polyethylene glycol moiety having from 1 to 10 PEG units. In an embodiment, the term “branched polyethylene glycol” means at least one H on the formula [CH₂CH₂O]ₙ— is substituted with substituent such as one or more of polyethylene glycol so as to form a branch structure.

Furthermore, L₂ is a cleavable linker or a non-cleavable linker. The non-cleavable liker includes, but are not limited to, a thioether linker and a maleimido caproyl linker. The cleavable liker includes, but are not limited to, a disulfide containing linker, an acid labile linker, a photolabile linker, a peptidase labile linker, an esterase labile linker, a phosphatase labile linker, a beta-glucuronide linker, a beta-glucuronidase labile linker, a beta-galactosidase labile linker and a sulfatase labile linker. The examples of L₂ include, but are not limited to, peptide-aminobenzylcarbamate linkers, e.g., maleimidocaproyl-L-phenylalanine-L-lysine-p-aminobenzylcarbamate and maleimidocaproyl-L-valine-L-citrulline-p-aminobenzylcarbamate (ve); N-succinimidyl 3-(2-pyridyldithio)proprionate (also known as N-succinimidyl 4-(2-pyridyldithio)pentanoate or SPP); 4-succinimidyl-oxycarbonyl-2-methyl-2-(2-pyridyldithio)-toluene (SMPT); N-succinimidyl 3-(2-pyridyldithio)propionate (SPDP); N-succinimidyl 4-(2-pyridyldithio)butyrate (SPDB); 2-iminothiolane; S-acetylsuccinic anhydride; disulfide benzyl carbamate; carbonate; hydrazone linkers; N-(a-Maleimidoacetoxy) succinimide ester; N-[4-(p-Azidosalicylamido) butyl]-3′-(2′-pyridyldithio)propionamide (AMAS); N-[b-Maleimidopropyloxy]succinimide ester (BMPS); [N-e-Maleimidocaproyloxy]succinimide ester (EMCS); N-[g-Maleimidobutyryloxy]succinimide ester (GMBS); Succinimidyl-4-[N-Maleimidomethyl]cyclohexane-1-carboxy-[6-amidocaproate] (LC-SMCC); Succinimidyl 6-(3-[2-pyridyldithio]-propionamido)hexanoate (LC-SPDP); m-Maleimidobenzoyl-N-hydroxysuccinimide ester (MBS); N-Succinimidyl[4-iodoacetyl]aminobenzoate (SIAB); Succinimidyl 4-[N-maleimidomethyl]cyclohexane-1-carboxylate (SMCC); N-Succinimidyl 3-[2-pyridyldithio]-propionamido (SPDP); [N-e-Maleimidocaproyloxy]sulfosuccinimide ester (Sulfo-EMCS); N-[g-Maleimidobutyryloxy]sulfosuccinimide ester (Sulfo-GMBS); 4-Sulfosuccinimidyl-6-methyl-a-(2-pyridyldithio)toluamido]hexanoate) (Sulfo-LC-SMPT); Sulfosuccinimidyl 6-(3′-[2-pyridyldithio]-propionamido)hexanoate (Sulfo-LC-SPDP); m-Maleimidobenzoyl-N-hydroxysulfosuccinimide ester (Sulfo-MBS); N-Sulfosuccinimidyl[4-iodoacetyl]aminobenzoate (Sulfo-SIAB); Sulfosuccinimidyl 4-[N-maleimidomethyl]cyclohexane-1-carboxylate (Sulfo-SMCC); Sulfosuccinimidyl 4-[p-maleimidophenyl]butyrate (Sulfo-SMPB); ethylene glycol-bis(succinic acid N-hydroxysuccinimide ester) (EGS); disuccinimidyl tartrate (DST); 1,4,7,10-tetraazacyclododecane-1,4,7,10-tetraacetic acid (DOTA); diethylenetriamine-pentaacetic acid (DTPA); thiourea linkers; glutamic acid-valine-citrulline; valine-Alanine; glutamic acid-valine-Alanine; valine-lysine; valine-lysine-glycine; Alanine-Alanine; Gly-Gly-Phe-Gly; Gly-Gly-Gly; β-glucuronide; β-galactoside; Pyrophosphate; phosphate; BrAc-Gly-Glu; CL2A; D-leucine-alanine-glutamate (DLAE); Furin-Cleavable Linker; L-Ala-D-Ala-L-Ala; Ortho Hydroxy-Protected Aryl Sulfate (OHPAS); and Val-Ser(GlcA).

L₃ is present or absent, and when present it connects the L₂ (when present) or reactive moieties G′ or connecting group CG with the payload. In a preffered embodiment, L₃ is a p-aminobenzylcarbamate (PAB).

The present disclosure also provides a method of treating cancer, comprising administrating an effective amount of the glycoprotein-payload conjugate comprising the structure of formula (1) as defined above to a subject in need thereof; a use of the glycoprotein-payload conjugate comprising the structure of formula (1) as defined above in the manufacture of a medicament for treating cancer; and a glycoprotein-payload conjugate comprising the structure of formula (1) as defined above for use in treating cancer in a subject in need thereof.

In at least one embodiment of the present disclosure, the cancer is selected from the group consisting of bladder cancer, bone cancer, brain tumor, breast cancer, colorectal cancer, eye melanoma, gastric carcinoma, head and neck cancer, kidney cancer, leukemia, lung cancer, lymphoma, melanoma, oral and oropharyngeal cancer, pancreatic cancer, prostate cancer, skin cancer, stomach cancer, thyroid cancer and uterine cancer, for example, the cancer can be breast cancer and/or gastric carcinoma.

The following examples are presented to illustrate certain embodiments of the present disclosure, but should not be construed as limiting the scope of this invention.

### EXAMPLE

Exemplary embodiments of the present disclosure are further described in the following examples, which should not be construed to limit the scope of the present disclosure.

### Example 1. Preparation of G0F/G0 Herceptin Antibody

To remove galactose and sialic acid moieties of the N-glycan from Herceptin antibody, which has a structure shown in formula (5), 10 mg of Herceptin antibody (Roche Inc.) was treated with 20 μl β1,4-galactosidase (NEB, P0745L, 8 unit/μl) and 5 μl α2-3,6,8 neuraminidase (NEB, P0720L, 50 unit/μl) in 1× GlycoBuffer 1 (NEB, total volume 1 ml) at 37° C. for 24 hours. 10 μl of 01,4-galactosidase (NEB, P0745L, 8 unit/μl) was further added to the reactant and the reaction was allowed to perform at 37° C. for another 24 hours to obtain G0F/G0 antibody sample, which has a structure shown in formula (6). The antibody sample was purified using rProtein A Sepharose Fast Flow (GE Healthcare, 17-1279-02). After purification, the antibody sample was subjected to Reduced Mass Chromatography Analysis. The results reveal that the major amount of the antibodies in the sample is G0F (having a heavy chain with a molecular weight of 50,600 Da) and only a small amount is G0 (without a fucose sugar; having a heavy chain with a molecular weight of 50,451 Da).

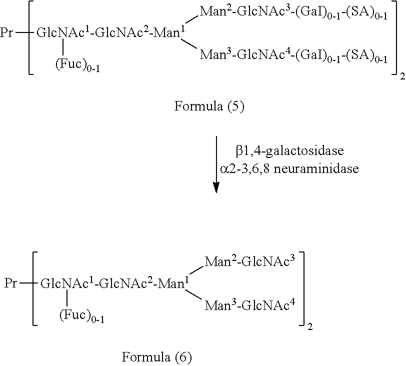

### Example 2. Preparation of Tri-Mannosyl Core Herceptin Antibody

10 mg of G0F/G0 Hercepin antibody from Example 1 (having a structure shown in formula (6) was treated with 20 μl β-N-Acetylglucosaminidase S (NEB, P0744L, 4 unit/μl) in 1× GlycoBuffer 1 (NEB, total volume 1 ml) at 37° C. for 24 hours. 10 μl of β-N-Acetylglucosaminidase S (NEB, P0744L, 4 unit/μl) was added to the reactant and the reaction was allowed to proceed at 37° C. for further 24 hours to obtain a digested antibody sample having a structure shown in formula (7). The digested antibody sample was purified by using rProtein A Sepharose Fast Flow (GE Healthcare, 17-1279-02). After purification, the antibody sample was subjected to reduced mass chromatography analysis. The tri-mannosyl core Herceptin antibody having a heavy chain with a molecular weight of 50,194 Da was obtained and that almost all of G0F and G0 Hercetin antibodies were converted to tri-mannosyl core antibodies. It suggests that β-N-Acetylglucosaminidase S is capable of converting G0F and G0 antibodies to ones having tri-mannosyl Core at a high efficiency.

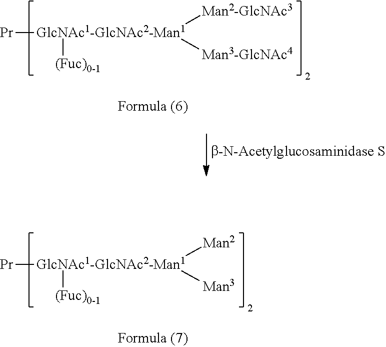

### Example 3. Transfer GlcNAz to Herceptin Antibody by GnT-3

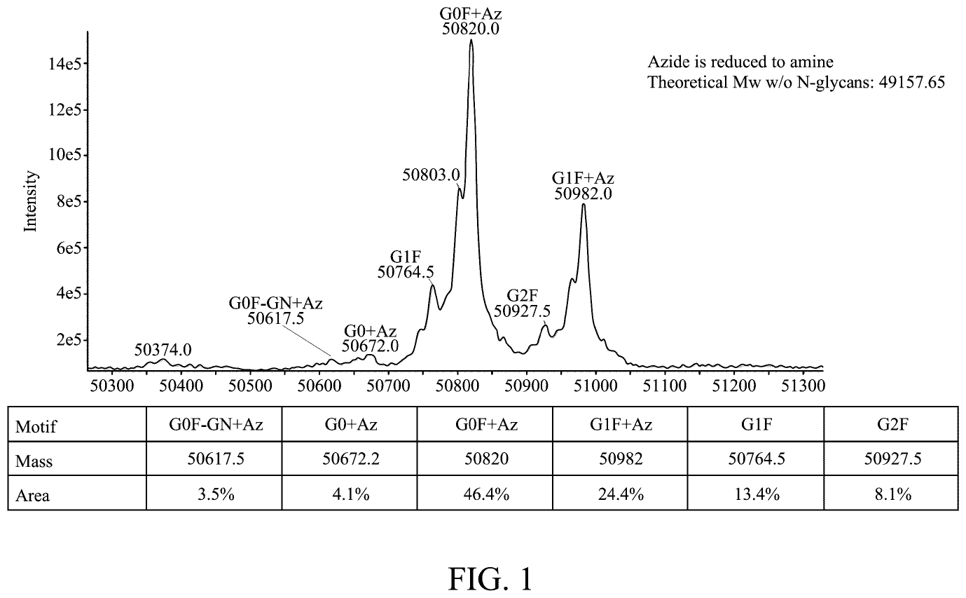

MGAT-3 transfers UDP-azido-N-acetylglucosamine to Man¹ of tri-mannosyl core protein. To confirm this phenomena in antibodies, Trimannosly-Herceptin obtained in Example 2 (25 μg) and UDP-GlcNAz (commerical from sci-pharmtech, CAS: 1611490-64-2) (16.6 μg) in 10 μl 1× buffer SP (25 mM MES (4-morpholineethanesulfonic acid), 10 mM MnCl₂, pH 6.5) were incubated in the presence of GnT-3 (0.75 μg; R&D, 7359-GT) at 37° C. for 18 hours. The produced protein was named Herceptin-2Az, which has a structure shown in formula (8). The protein was subjected to a reduced mass chromatography analysis. The result is shown in FIG. 1.

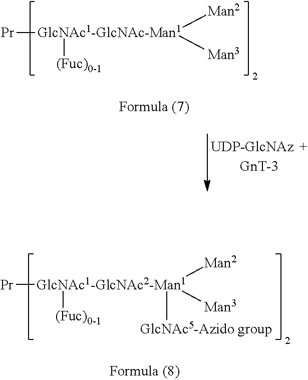

### Example 4. Transfer GlcNAz to Tri-Mannosyl Core Herceptin Antibody by GnT-1, GnT-2 and GnT-3

Trimannosly-Herceptin obtained in Example 2 (5 mg) and UDP-GlcNAz (commerical from sci-pharmtech, CAS: 1611490-64-2) (2.5 mg) in 800 μl 1× buffer SP (25 mM MES, 10 mM MnCl₂, pH 6.5) were incubated in the presence of GnT-1 (production by Development center of Biotechnology) (0.2 mg), GnT-2 (production by Development center of Biotechnology) (0.05 mg) and GnT-3 (0.05 mg; R&D, 7359-GT) at 37° C. for 16 hours. After the incubation, the produced protein was named Herceptin-6Az, which has a structure shown in formula (9). The protein was subjected to a reduced mass chromatography analysis.

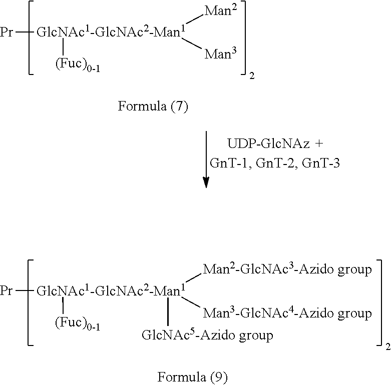

### Example 5. Conjugate Herceptin-2Az with Linker-Payload

In this Example, two kinds of therapeutic agents, exatecan and MMAE, were used as the payload of interest. The two ADCs produced by conjugating Herceptin-2Az obtained in Example 3 with the payloads were prepared as the following:

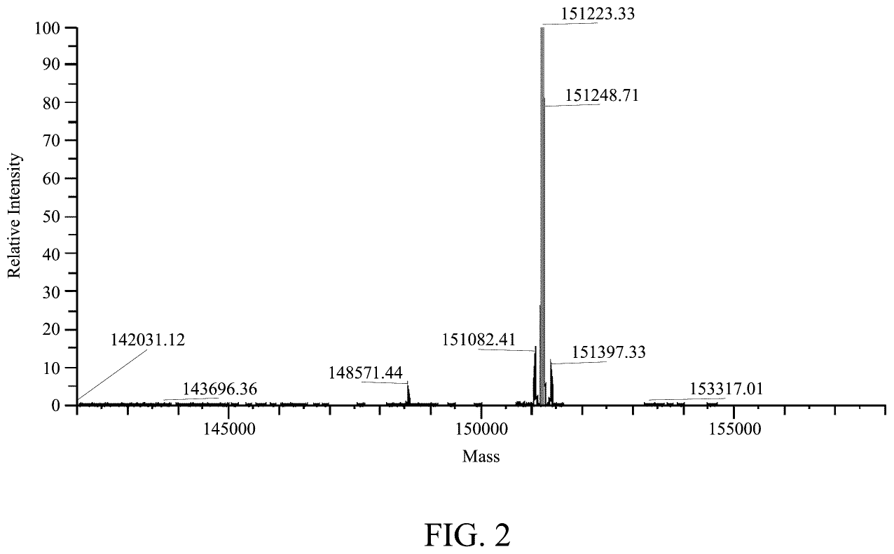

DBCO-PEG3-vc-exatecan: 10 equivalents of DBCO-PEG3-vc-exatecan (10 mM DMA solution,) was slowly added to the Herceptin-2Az protein solution obteained in Example 3 (0.5 mg antibody, 2.366 mg/mL) in buffer (80 mM Na-citrate, 0.18 M Tris, pH 6.5). The reaction mixture was incubated in shaking incubator at 37° C. for 18 hours. Amicon Ultra-15 centrifugal filter device with 30 kDa NMWL and 25 mM Na-citrate pH 6.5 buffer were used for purification and concertation of ADC. The ADC thus obtained was named Herceptin-2exatecan, which has a structure shown in formula (10). The ADC concentration was estimated by measuring the absorbance at 280 nm and used for calculation of recovery yield: 86%. The drug-to-antibody ratio (DAR) of ADC was measured by LC-MS: 1.95. The obtained LC-MS spectrum is shown in FIG. 2.

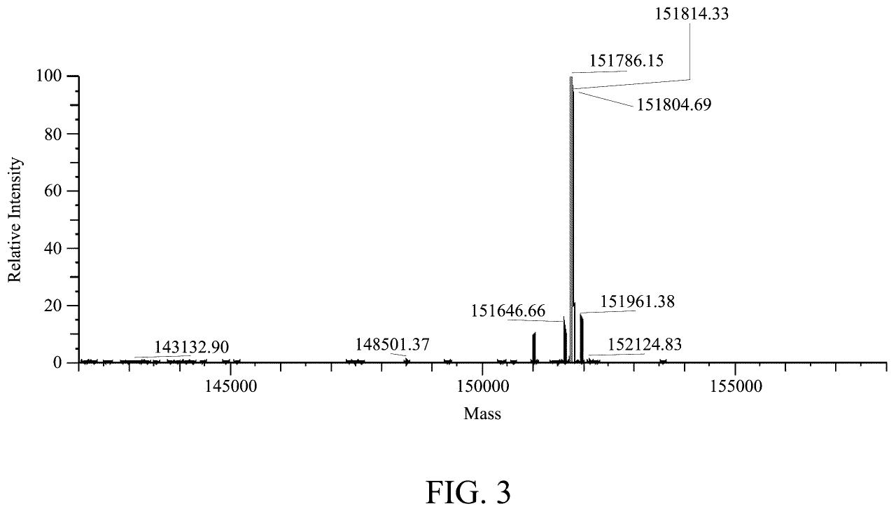

DBCO-PEG3-vc-MMAE: 15 equivalents of DBCO-PEG3-vc-MMAE (10 mM DMA solution,) was slowly added to the Herceptin-2Az protein solution obteained in Example 3 (0.71 mg antibody, 3.449 mg/mL) in buffer (25 mM Na-citrate pH 6.5). The reaction mixture was incubated in shaking incubator at 37° C. for 18 hours. Amicon Ultra-15 centrifugal filter device with 30 kDa NMWL and 25 mM Na-citrate pH 6.5 buffer were used for removal of DBCO-PEG3-vc-MMAE. The ADC thus obtained was named Herceptin-2MMAE, which has a structure shown in formula (10). The ADC concentration was estimated by measuring the absorbance at 280 nm and used for calculation of recovery yield: 81%. The drug-to-antibody ratio (DAR) of ADC was measured by LC-MS: 1.94. The obtained LC-MS spectrum is shown in FIG. 3.

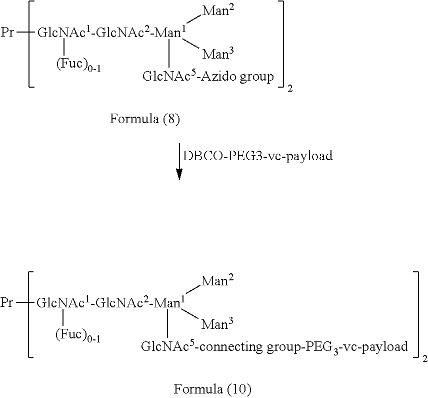

### Example 6. Conjugate Herceptin-6Az with Linker-Payload

In this Example, two kinds of therapeutic agents, exatecan and MMAE, were used as the payload of interest. The two ADCs produced by conjugating Herceptin-6Az obtained in Example 4 with the payloads were prepared as the following:

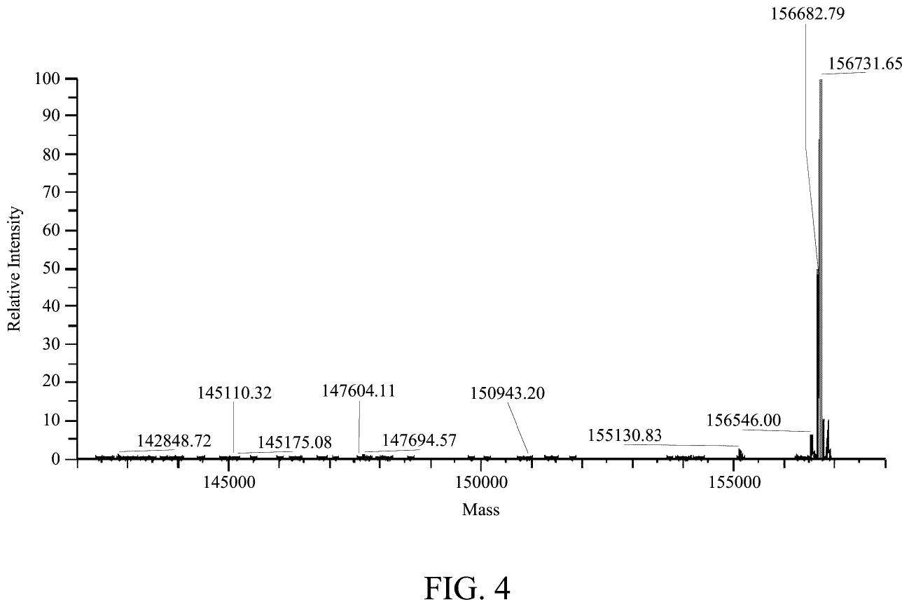

DBCO-PEG3-vc-exatecan: 15 equivalents of DBCO-PEG3-vc-exatecan (10 mM DMA solution) was slowly added to the Herceptin-6Az protein solution obteained in Example 4 (0.5 mg antibody, 2.1 mg/mL) in buffer (80 mM Na-citrate, 0.21 M Tris, pH 6.5). The reaction mixture was incubated in shaking incubator at 37° C. for 18 hours. Amicon Ultra-15 centrifugal filter device with 30 kDa NMWL and 25 mM Na-citrate pH 6.5 buffer were used for removal of DBCO-PEG3-vc-exatecan. The ADC thus obtained was named Herceptin-6exatecan, which has a structure shown in formula (11). The ADC concentration was estimated by measuring the absorbance at 280 nm and used for calculation of recovery yield: 67%. The drug-to-antibody ratio (DAR) of ADC was measured by LC-MS: 5.99. The obtained LC-MS spectrum is shown in FIG. 4.

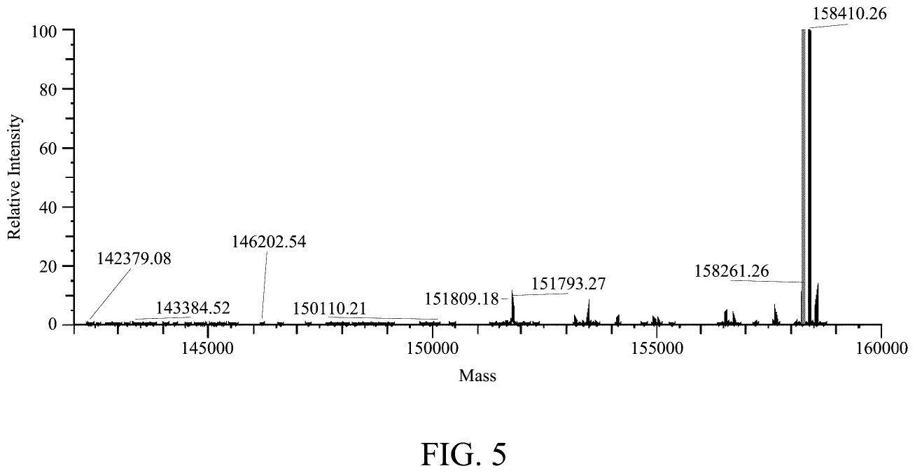

DBCO-PEG3-vc-MMAE: 30 equivalents of DBCO-PEG3-vc-MMAE (10 mM DMA solution) was slowly added to the Herceptin-6Az protein solution obteained in Example 4 (1.0 mg antibody, 2.1 mg/mL) in buffer (80 mM Na-citrate, 0.2 M Tris, pH 6.0). The reaction mixture was incubated in shaking incubator at 37° C. for 18 hours. Amicon Ultra-15 centrifugal filter device with 30 kDa NMWL and 25 mM Na-citrate pH 6.5 buffer were used for removal of DBCO-PEG3-vc-MMAE. The ADC thus obtained was named Herceptin-6MMAE, which has a structure shown in formula (11). The ADC concentration was estimated by measuring the absorbance at 280 nm and used for calculation of recovery yield: ˜71%. The drug-to-antibody ratio (DAR) of ADC was measured by LC-MS: 5.62. The obtained LC-MS spectrum is shown in FIG. 5.

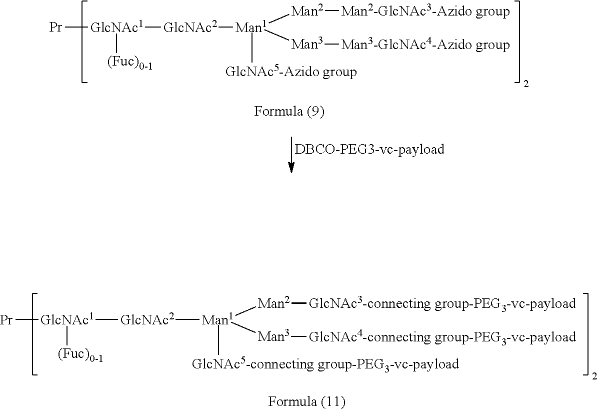

### Example 7. Cytotoxicity of Herceptin ADCs in BT-474 and NCI-N87 Cells

In this Example, two kinds of cancer cell lines were used for testing the cytotoxicities of the ADCs respectively obtained in the above Examples 5 and 6.

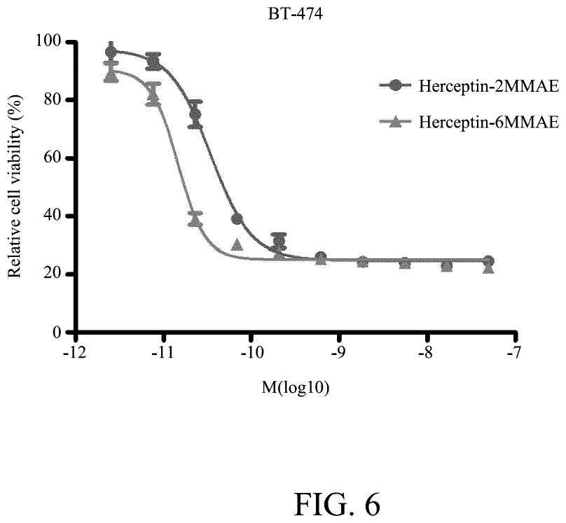

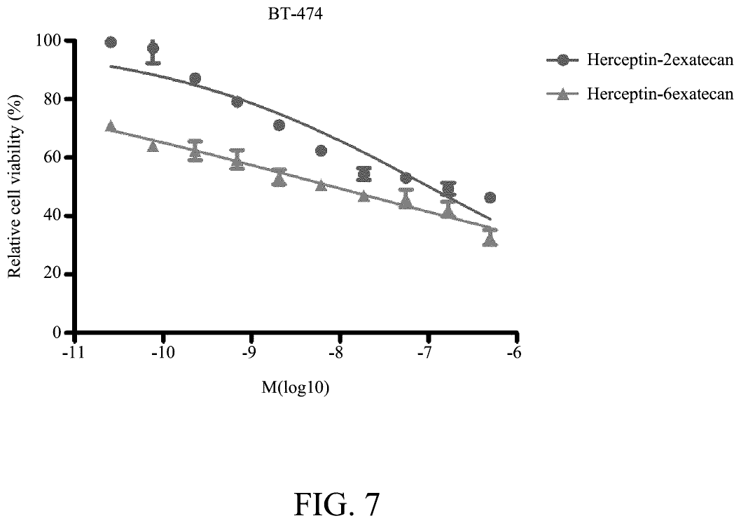

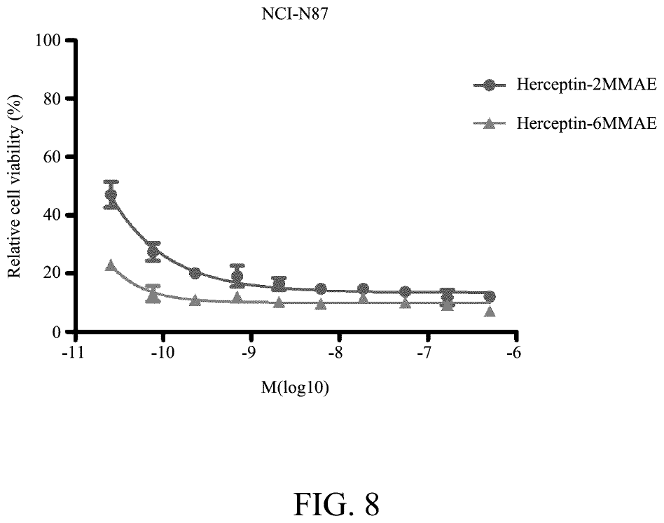

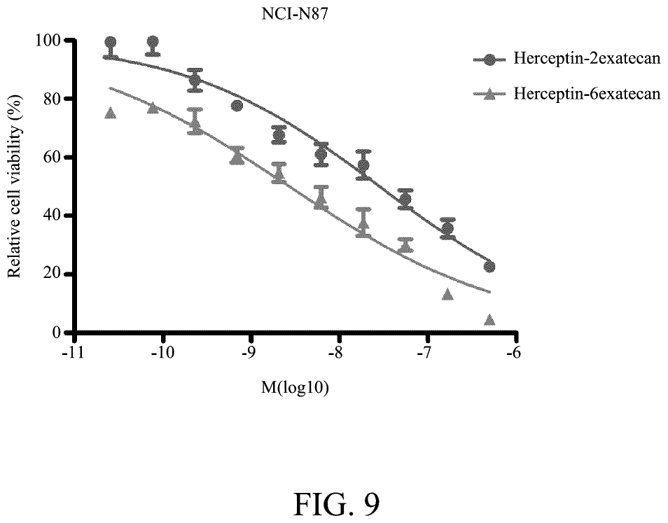

BT-474, a HER2 positive human breast cancer cell line, was obtained from the ATCC. This cell line was cultured in Hybri-Care Medium (ATCC) which is supplied as a powder and should be reconstituted in 1 L cell-culture-grade water and supplemented with 1.5 g/L sodium bicarbonate and 10% fetal bovine serum (Gibco). NCI-N87, a HER2 positive human gastric carcinoma cell line, was obtained from the ATCC. This cell line was cultured in in RPMI (ATCC), supplemented with 10% fetal bovine serum (Gibco). The BT-474 and NCI-N87 cell lines were maintained in an atmosphere of 5% CO₂ in a humidified 37° C. incubator. The day before treatment, cells were collected and seeded into 96-well plates (BT-474: 2,500 cells per well, NCI-N87: 1,500 cells per well). On the second day, cells were treated with 3-fold serial dilution concentration of ADCs obtained in the above Examples 5 and 6, respectively. Each treatment was performed in ten triplicate data points. After the treatment of 144 hours, cell viability was assessed by Cell Titer-Glo kit (Promega) according to the manufacturer's instruction. At the end of the incubation, luminescence was measured using a SpectraMax i3x Multi Mode Detection Platform (Molecular Devices). Compound cytotoxicity was evaluated in comparison to cells treated with 0.05% PBS (ADCs) or 0.05% DMSO (toxic payload). IC₅₀ values were calculated by fitting viability data with a four-parameter logistic equation using GraphPad prism 5.0 software. The results are shown in Table 1 and FIGS. 6-9.

TABLE 1
IC₅₀ values of Herceptin ADCs.

Cytotoxicity (IC50) of ADCs (nM)

| ADC name | BT-474 | NCI-N87 |
|---|---|---|
| Herceptin-2MMAE | 0.0344 | <0.1 |
| Herceptin-6MMAE | 0.0146 | <0.1 |
| Herceptin-2exatecan | 101.2 | 28.3 |
| Herceptin-6exatecan | 8.4 | 2.7 |

As shown in the Table 1 and FIGS. 6-9, the glycoprotein-conjugates of the present disclosure having specific structure, no matter with 2 or 6 therapeutics thereon, can provide an excellent inhibition effect on cancer cells, and thus can be used for treating cancers.

Furthermore, it is further found that the level of the inhibition effect of the glycoprotein-conjugates of the present disclosure can be adjusted by changing the number of payloads conjugated thereon, indicating that the drug antibody ratios (DAR) and the payload diversity of the glycoprotein-conjugates of the present disclosure can be designed precisely according to the clinical needs, for example, the number of payloads can be designed according to the severity and type of the disease; or the age, condition and gender of the patient.

The above-described descriptions of the detailed embodiments are to illustrate the preferred implementation according to the present disclosure, and it is not to limit the scope of the present disclosure. Accordingly, all modifications and variations completed by those with ordinary skill in the art should fall within the scope of the present disclosure defined by the appended claims.

What is claimed is:

1. A method for preparing a glycoprotein-conjugate comprising a structure of formula (1) 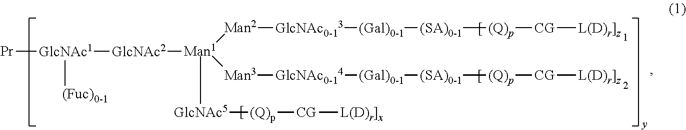
wherein:
Pr is a glycoprotein;
L is a linker;
D is a payload of interest;
x is 1, 2, 3, or 4;
y is 1 or 2;
z₁ is 0 or 1;
z₂ is 0 or 1;
r is a positive integer from 1-20;
p is an integer from 0-8;
Q is alkylene, alkenylene or polyehylene glycol;
CG is a connecting group obtainable by a reaction of C′ and G′;
C′ is or comprises a reative group selected from an azido group, a keto group, an alkynyl group, a thiol group, a halogen, a sulfonyloxy group, a halogenated acetamido group, a mercaptoacetamido group, a sulfonylated hydroxyacetamido group, a cyclopropenyl group, a trans cyclooctene group, a cycloalkyne group, a tetrazyinyl group, a maleimide group, a cyclononyne moiety and a cyclooctyne moiety; G′ is or comprises a reactive group capable of undergoing a reaction with C′;
provided that when z₁ is 1, GlcNAc³ is present, and galactose (Gal) and sialic acid (SA) connected thereon do not present;
provided that when z₂ is 1, GlcNAc⁴ is present, and galactose (Gal) and sialic acid (SA) connected thereon do not present,
the method comprising the steps of:
(i) reacting a glycoprotein comprising a structure of formula (1-1): 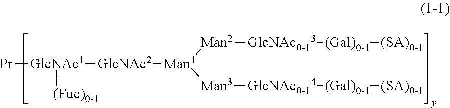
with a molecule of UDP-GlcNAc-(Q)₀₋₈-C′ in the presence of beta-1,4-mannosyl-glycoprotein 4-beta-N-acetylglucosaminyltransferase and optionally mannosyl (α-1,3-)-glycoprotein β-1,2-N-acetylglucosaminyltransferase and optionally mannosyl (α-1,6-)-glycoprotein β-1,2-N-acetylglucosaminyltransferase to bond a molecule of GlcNAc-(Q)₀₋₈-C′ to Man¹ and optionally Man² and Man³; and thereby forming a glycoprotein comprising a glycan of formula (1-2): 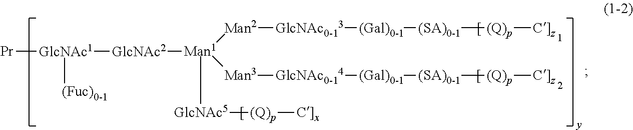
and
(ii) reacting the glycoprotein comprising the glycan of formula (1-2) with one or more linker-payload conjugates comprising a structure of G′-L(D)ᵣ, thereby forming the glycoprotein-payload conjugate comprising the structure of formula (1).

2. The method of claim 1, wherein the glycoprotein is an antibody or a fragment thereof.

3. The method of claim 1, wherein G′ comprises a terminal azido, alkyne, cyclononyne moiety, or a cyclooctyne moiety, and wherein G′ is different from C′.

4. The method of claim 1, wherein the cyclononyne moiety is bicyclononyne (BCN) and the cyclooctyne moiety is selected from the group consisting of azadibenzocyclooctyne (DIBAC/DBCO), dibenzocyclooctyne (DIBO) and sulfonylated dibenzocyclooctyne (s-DIBO).

5. The method of claim 1, wherein the linker comprises a structure of L₁-L₂-L₃, wherein,
L₁ is present or absent, and when present it is a spacer;
L₂ is a cleavable linker or a non-cleavable linker; and
L₃ is present or absent, and when present it is a PAB.

6. The method of claim 5, wherein L₁ is a linear or branched PEG chain having 1 to 10 PEG units, alkylene, cycloalkylene, alkenylene, cycloalkenylene, alkynylene, arylene, heteroarylene, alkeneoxy, acyl, alkylamines, or arylamine group having 2 to 20 carbon atoms;
and/or L₂ is a thioether linker, a maleimido caproyl linker, a disulfide containing linker, an acid labile linker, a photolabile linker, a peptidase labile linker, an esterase labile linker, a phosphatase labile linker, a beta-glucuronide linker, a beta-glucuronidase labile linker, a beta-galactosidase labile linker or a sulfatase labile linker.

7. The method of claim 5, wherein L₂ is selected from the group consisting of peptide-aminobenzylcarbamate linkers; L-phenylalanine-L-lysine-p-aminobenzylcarbamate and L-valine-L-citrulline-p-aminobenzylcarbamate (vc); N-succinimidyl 3-(2-pyridyldithio)proprionate; 4-succinimidyl-oxycarbonyl-2-methyl-2-(2-pyridyldithio)-toluene (SMPT); N-succinimidyl 3-(2-pyridyldithio)propionate (SPDP); N-succinimidyl 4-(2-pyridyldithio)butyrate (SPDB); 2-iminothiolane; S-acetylsuccinic anhydride; disulfide benzyl carbamate; carbonate; hydrazone linkers; N-(a-Maleimidoacetoxy) succinimide ester; N-[4-(p-Azidosalicylamido) butyl]-3′-(2′-pyridyldithio)propionamide (AMAS); N-[b-Maleimidopropyloxy]succinimide ester (BMPS); [N-e-Maleimidocaproyloxy]succinimide ester (EMCS); N-[g-Maleimidobutyryloxy]succinimide ester (GMBS); Succinimidyl-4-[N-Maleimidomethyl]cyclohexane-1-carboxy-[6-amidocaproate] (LC-SMCC); Succinimidyl 6-(3-[2-pyridyldithio]-propionamido)hexanoate (LC-SPDP); m-Maleimidobenzoyl-N-hydroxysuccinimide ester (MBS); N-Succinimidyl[4-iodoacetyl]aminobenzoate (SIAB); Succinimidyl 4-[N-maleimidomethyl]cyclohexane-1-carboxylate (SMCC); N-Succinimidyl 3-[2-pyridyldithio]-propionamido (SPDP); [N-e-Maleimidocaproyloxy]sulfosuccinimide ester (Sulfo-EMCS); N-[g-Maleimidobutyryloxy]sulfosuccinimide ester (Sulfo-GMBS); 4-Sulfosuccinimidyl-6-methyl-a-(2-pyridyldithio)toluamido]hexanoate) (Sulfo-LC-SMPT); Sulfosuccinimidyl 6-(3′-[2-pyridyldithio]-propionamido)hexanoate (Sulfo-LC-SPDP); m-Maleimidobenzoyl-N-hydroxysulfosuccinimide ester (Sulfo-MBS); N-Sulfosuccinimidyl[4-iodoacetyl]aminobenzoate (Sulfo-SIAB); Sulfosuccinimidyl 4-[N-maleimidomethyl]cyclohexane-1-carboxylate (Sulfo-SMCC); Sulfosuccinimidyl 4-[p-maleimidophenyl]butyrate (Sulfo-SMPB); ethylene glycol-bis(succinic acid N-hydroxysuccinimide ester) (EGS); disuccinimidyl tartrate (DST); 1,4,7,10-tetraazacyclododecane-1,4,7,10-tetraacetic acid (DOTA); diethylenetriamine-pentaacetic acid (DTPA); thiourea linkers; glutamic acid-valine-citrulline; valine-Alanine; glutamic acid-valine-Alanine; valine-lysine; valine-lysine-glycine; Alanine-Alanine; Gly-Gly-Phe-Gly; Gly-Gly-Gly; β-glucuronide; β-galactoside; Pyrophosphate; phosphate; BrAc-Gly-Glu; CL2A; D-leucine-alanine-glutamate (DLAE); Furin-Cleavable Linker; L-Ala-D-Ala-L-Ala; Ortho Hydroxy-Protected Aryl Sulfate (OHPAS); and Val-Ser(GlcA).

8. The method of claim 1, wherein the payload is a therapeutic agent selected from antimetabolites, alkylating agents, alkylating-like agents, DNA minor groove alkylating agents, anthracyclines, antibiotics, calicheamicins, antimitotic agents, topoisomerase inhibitors, proteasome inhibitors, and radioisotopes.

9. The method of claim 8, wherein the therapeutic agent is selected from exatecan and MMAE.

10. The method of claim 1, wherein the payload is a label selected from a fluorescent label, a chromophoric label, an electron-dense label, a chemiluminescent label, a radioactive label, an enzymatic label, or a positron emitter.

11. The method of claim 1, which further comprises adding a β-N-acetylglucosaminidase before conducting the step (i) to remove GlcNAc on the glycoprotein of formula (1-1).

12. A glycoprotein-payload conjugate comprising the structure of formula (1) as defined in claim 1.

13. The glycoprotein-payload conjugate of claim 12, which has the following formula (2):

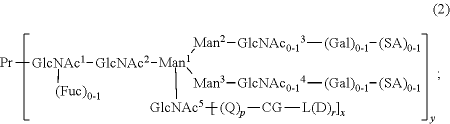

14. The glycoprotein-payload conjugate of claim 12, which has the following formula (3): 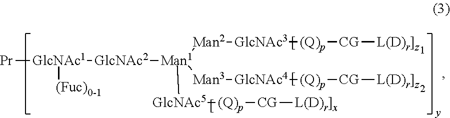
wherein, z₁ and z₂ are both 1, or z₁ is 1 and z₂ is 0.

15. The glycoprotein-payload conjugate of claim 12, wherein the payload is a therapeutic agent selected from antimetabolites, alkylating agents, alkylating-like agents, DNA minor groove alkylating agents, anthracyclines, antibiotics, calicheamicins, antimitotic agents, topoisomerase inhibitors, proteasome inhibitors, and radioisotopes.

16. The glycoprotein-payload conjugate of claim 15, wherein the therapeutic agent is selected from exatecan and MMAE.

17. The glycoprotein-payload conjugate of claim 15, wherein the payload is a label selected from a fluorescent label, a chromophoric label, an electron-dense label, a chemiluminescent label, a radioactive label, an enzymatic label, or a positron emitter.

18. A method of treating cancer, comprising administrating an effective amount of the glycoprotein-payload conjugate of claim 12 to a subject in need thereof.

19. The method of claim 18, wherein the cancer is selected from the group consisting of bladder cancer, bone cancer, brain tumor, breast cancer, colorectal cancer, eye melanoma, gastric carcinoma, head and neck cancer, kidney cancer, leukemia, lung cancer, lymphoma, melanoma, oral and oropharyngeal cancer, pancreatic cancer, prostate cancer, skin cancer, stomach cancer, thyroid cancer and uterine cancer.

20. The method of claim 19, wherein the cancer is breast cancer and/or gastric carcinoma.
